(19) United States
(12) Patent Application Publication
(10) Pub. No.: US 2026/0191229 A1
(43) Pub. Date: Jul. 9, 2026

(54) Process for preserving and transforming algae by lacto-fermentation, use of the lacto-fermented juice and algae obtained according to the process

(71) Applicant: ALGROUPE, Tregueux (FR)

(72) Inventor: Stéphane Mahe, Saint Brieuc (FR)

(21) Appl. No.: 19/133,992

(22) PCT Filed: Nov. 30, 2023
(86) PCT No.: PCT/EP2023/083753

§ 371 Date: May 29, 2025

(30) Foreign Application Priority Data
Dec. 1, 2022 (FR) FR2212610

(51) Int. Cl. A23K 10/12; A23K 10/18; A23K 50/10; A23K 50/30; A23K 50/75; A23L 17/60; A61K 8/9706; C12N 1/12; C12N 1/20; C12R 1/225; C12R 1/89

(52) U.S. Cl. CPC A23K 10/12; A23K 10/18; A23K 50/10; A23K 50/30; A23K 50/75; A23L 17/60; A61K 8/9706; C12N 1/12; C12N 1/20; A61K 2800/85; C12R 2001/225; C12R 2001/89

(57) ABSTRACT

The invention essentially consists of a process for the lacto-fermentation of fresh algae which can be natural or with inoculation with pH control and advantageously under anaerobic conditions and which makes it possible to produce concomitantly ready-to-eat algae in vegetable form and a fermentation juice which can be drunk directly by animals or mixed with animal feed. After the pH is stabilized, the algae and fermented juice that have been obtained are separated.

### TECHNICAL FIELD

The present invention relates generally to the field of preserving and transforming algae for use notably in human, animal or plant food or in cosmetics.

It relates to a new process of transforming freshly harvested algae by lacto-fermentation, particularly in the marine environment.

The main aim of the invention is to provide a simple, fast and as natural a process as possible for optimally extracting the natural properties of algae, notably of marine algae.

All types of marine algae are suitable for the invention. These include: Gracilaria verrucosa, Chondrus crispus, Himanthalia elongata, commonly known as sea beans, Laminaria saccharina, Laminaria digitata, Undaria pinnatifida, Palmaria palmata, Ulva lactuca, commonly known as sea lettuce, Ulva armoricana, Ulva intestinalis, Solieria Chordalis, Porphyra umbilicalis, Fucus visiculosus, Ascophyllum nodosum, Saccharina Japonica, commonly known as kombu seaweed, Sargassum, Macrocystis pyrifera, Asparagopsis taxiformis, Alaria esculenta, Alaria marginata, commonly known as wakame.

### PRIOR ART

The qualities of fresh algae have already been recognized for food in general, as human food or animal feed, or for cosmetics and for plant treatment. Thus, algae is already harvested and consumed by people and animals all over the world and is also used as a soil conditioner, for example in the form of seaweed.

Algae is also harvested or cultivated for the extraction of alginate, agar and carrageenan, gelatinous substances collectively known as hydrocolloids (mainly used as thickeners in food, pharmaceutical and nutraceutical preparations, etc.) or phycocolloids for use as food additives. The food industry exploits their gelling, water retention, emulsifying and other physical properties. Agar is widely used in foods such as confectionery, meat and poultry-based products, desserts and beverages and molded foods. Carrageenan is used in dressings and sauces, health foods and as a preservative in meat and fish-based products, dairy products and bakery products.

Additionally, marine algae is already sought after and exploited for its high nutritional content in vitamins, proteins, oligo elements, omega 3 and 6 fatty acids beneficial for health, etc. Algae as a vegetable has been approved for consumption in France since the early 1980s. Only a limited number of “vegetable” algae varieties benefit from this approval. Their sale is subject to strict regulatory requirements, mainly concerning their heavy metal and iodine content.

To date, edible algae has mainly been stabilized by brining or drying.

Ensiling or lacto-fermentation has already been implemented for freshly harvested marine algae, as for example described in application WO2013/045931. Ensiling or lacto-fermentation is a traditional vegetable process that can therefore be applied to algae and thus aims to preserve it over long periods while providing organoleptic (texture, taste) and nutritional benefits.

The finished products are interesting for several reasons notably in terms of taste: lacto-fermented algae is tender, slightly crunchy and very pleasant in texture and in taste. Additionally, lacto-fermented algae retains all of its vitality and nutritional properties notably its high magnesium and vitamin C content. The properties of lactic acid bacteria also improve the digestibility thereof. The transformation performed by bacteria improves digestibility.

Compared with other processes of transforming fresh algae that have been developed in the past, including drying followed by dehydration, storage in saturated brine, bleaching followed by salting, and treatment by mixing with gray ash, the major advantages of a lacto-fermentation process are that any undesirable pathogenic micro-organisms present in the natural biotope are destroyed during fermentation, and that lactic acid bacteria are not only preserved, but also developed in their growth.

However, there is a need to further improve the processes for the lacto-fermentation of fresh algae for consumption as a food product, notably to enable algae to be preserved over a long period without rotting and without uncontrolled changes to its texture or external appearance, particularly its color.

Moreover, there is a growing demand for more effective and/or reliable animal feeds than those currently available. Alternatives to the synthetic products used in livestock farming and in agriculture are also sought.

There is therefore a further need to find a solution for adapting the lacto-fermentation of fresh algae to provide natural animal feeds that are more effective and/or reliable than those currently available.

The aim of the invention is to meet this (these) need(s) at least in part.

### DISCLOSURE OF THE INVENTION

To this end, the invention relates to a process for transforming algae comprising the following steps:
i) providing algae harvested from aquatic environments, frozen if necessary;
ii) washing the algae provided according to step i) with water;
iii) optionally, cutting or grinding the algae washed according to step ii);
iv) optionally, providing an inoculum comprising predominantly lactic acid-producing bacteria;
v) either inoculating the algae optionally cut or ground according to step iii) with the inoculum provided according to step iv) which comprises a concentration of lactic acid bacteria sufficient for the growth of the bacteria present in the algae so as to perform the lacto-fermentation thereof, or natural lacto-fermentation of the algae until the measured pH of the lacto-fermented composition has fallen below a predetermined final threshold;
vi) collecting at least part of the lacto-fermented juice obtained, for human, plant or animal use;
vii) washing the lacto-fermented algae, followed by its storage and collection for human, plant or animal use.

The algae provided in step i) is preferably marine algae selected from one of the following species: Gracilaria verrucosa, Chondrus crispus, Himanthalia elongata, commonly known as sea beans, Laminaria digitata, commonly known as Breton kombu seaweed, Undaria pinnatifida, Palmaria palmata, Ulva lactuca, commonly known as sea lettuce, Ulva armoricana, Ulva intestinalis, Solieria Chordalis, Porphyra umbilicalis, Fucus visiculosus, Ascophyllum nodosum, Saccharina Japonica, commonly known as kombu seaweed, Saccharina latissima, commonly known as Royal Kombu algae, Sargassum, Macrocystis pyrifera, Asparagopsis taxiformis, Undaria pinnatifida, Alaria esculenta, Alaria marginata, commonly known as wakame or a combination thereof.

Washing step ii) is advantageously performed by soaking and bubbling. This improved washing must be considered particularly for washed up algae, that is, which has been detached/torn off from its natural support and is harvested when it notably washes up on a beach. This gets rid of unwanted sand, pebbles and plastic waste. For hand-harvested algae, simple cleaning with water may suffice.

This washing step ii) can comprise a bleaching sub-step for fresh algae to reduce endogenous flora and to promote the development of the subsequent inoculation.

Step iii) is advantageously carried out so that the length of the cut algae is between 0.2 and 5 cm. Based on the type of algae, grinding increases the contact surface between the algae and the ferments and therefore promotes the fermentation of the algae.

For marine algae, the cut length is preferably between:
2 and 5 cm for Himanthalia elongata commonly known as sea beans,
0.2 and 1 cm for Saccharina latissima, commonly known as Royal Kombu algae,
0.2 and 1 cm for Laminaria digitata,
0.5 to 2 cm for Undaria pinnatifida.

The lactic acid bacteria are preferably selected from one of the following species: Lactobacillus plantarum, Leuconostoc mesenteroides, Lactobacillus lactis, Lactobacillus zeae, Lactobacillus casei or paracasei, Lactobacillus harbinensis, Leuconostoc kimch, Lactobacillus delbrueckii, Lactobacillus rhamnosus, Lactobacillus harbinensis, Streptococcus thermophilus, Propionibacterium or a combination thereof.

Advantageously, inoculation step v) is carried out using a combination of Leuconostoc mesenteroides and Lactobacillus lactis or zeae.

Even more advantageously, the inoculation rate is between 10⁵ and 10⁷ CFU/g. Such a rate is a very good compromise between the desired inoculation efficacy and the production cost.

According to one advantageous embodiment, the process comprises, prior to inoculation step v), packaging the cut or ground algae in a storage tank followed by anaerobic digestion.

According to an advantageous alternative embodiment, collection step vi) is carried out by racking the lacto-fermented juice.

According to this alternative, the process comprises, prior to step vii), a step of adding a liquid selected from water, water and acid or a combination thereof, the volume of liquid added being substantially equal to that of the racked lacto-fermented juice.

Preferably, once the liquid has been added and before racking, the process comprises a maceration step lasting between 1 and 20 days, preferably between 1 and 15 days.

In the process according to the invention, the predetermined final pH threshold is preferably less than or equal to 4.5.

An advantageous alternative consists, once the juice has been collected according to step iv), of a bleaching step on the lacto-fermented algae, still in their moist state so as to stabilize them. Bleaching agents such as water baths, with acids where appropriate, combined with a temperature rise where appropriate, can act by oxidation or by reduction. This bleaching step can reduce the content of certain elements that may be the most soluble after fermentation. To recover them, it is possible to consider filtering the bleach bath.

Another object of the invention is the use of lacto-fermented algae collected in step vii) of the process as described previously, for human consumption.

The invention also covers the use of the lacto-fermented juice collected in step vi) of the process as described previously, as a palatability enhancer for animal feed, notably for sheep, cattle or pigs or poultry, as a colorant, flavoring and/or foaming agent, or as an emulsifier in human food or animal feed and in cosmetics.

The invention also covers the use of the lacto-fermented juice collected in step vi) of the process as described previously, optionally mixed with lacto-fermented algae collected in step vii) of the process as described previously as a phytostimulant for plant production and/or for improving soil quality. For example, it is possible to use the lacto-fermented juice or a mixture with lacto-fermented algae to spread topsoil or be sprayed directly onto the soil.

The invention also covers the use of lacto-fermented juice collected in step vi) of the process as described previously, in pharmacology.

Thus, the invention essentially consists of a process for the lacto-fermentation of fresh algae which can be natural or with inoculation with pH control and advantageously under anaerobic conditions and which makes it possible to produce concomitantly ready-to-eat algae in vegetable form and a fermentation juice which can be drunk directly by animals or mixed with animal feed. After the pH is stabilized, the algae and fermented juice that have been obtained are separated.

Lacto-fermentation may be natural or require inoculation based on the type of algae.

Indeed, the inventor has observed that certain species of algae, such as Royal Kombu, undergo natural lacto-fermentation as soon as the algae is subjected to a certain heat. The change in texture indicates that fermentation has already begun. This observation can be corroborated by measuring the pH, notably after grinding a sample. This is lacto-fermentation as the inventor was able to observe the production of acid. Typically, under certain heat conditions, the inventor has been able to observe that Royal Kombu ferments naturally within two days.

Inoculation can be used to accelerate or trigger natural lacto-fermentation.

The length of the lacto-fermentation process is adapted based on the type of fresh algae to be processed, the desired final texture thereof and the juice to be obtained. For the latter, color, odor and/or viscosity are indicators of the degree of final fermentation.

During the process, the algae can be cut or ground.

Cutting increases the contact surface area for fermentation between algae and ferments.

Grinding is preferred when trying to obtain large quantities of juice. For Ulva lactuca, commonly known as sea lettuce, it is possible to dispense with the cutting step.

By virtue of the lacto-fermentation process according to the invention, co-products are systematically and directly obtained, that is, ready-to-eat algae in lacto-fermented vegetable form and a juice notably that can be consumed directly by animals. As previously stated, once collected, these co-products can be blended for direct use in human, plant or animal feed.

According to the inventor, the results of obtaining the nutritional qualities of algae and juices are highly reproducible for a very large number of algae varieties notably marine algae.

The invention just described has numerous advantages, including:
a simple, fast and as natural a process as possible for optimally extracting the natural properties of algae, notably marine algae, for a wide range of applications of the juice and/or lacto-fermented algae ultimately obtained;
an “eco-responsible” process, as it requires very little energy notably because there is no need to generate cold, as all the steps of the process can be carried out at room temperature.
an “eco-responsible” process that allows the algae to be stabilized and stored for several months while preserving the properties thereof.

Further advantages and features will become apparent from reading the detailed description, which is illustrative and non-limiting, with reference to the following figures.

### BRIEF DESCRIPTION OF THE DRAWINGS

FIGS. 1A and 1B show an overview of the essential steps of an algae lacto-fermentation process according to the invention.

FIG. 2 shows a longitudinal sectional view of an example of a fermentation tank used in the lacto-fermentation process according to the invention.

FIGS. 3 and 4 show in curve form the monitoring of the fermentation pH of an example of algae, respectively Himanthalia elongata and Saccharina during a process according to the invention.

### DETAILED DESCRIPTION

Throughout the application, the terms “inlet”, “outlet”, “upstream”, “downstream” are to be understood in relation to the direction of circulation of the algae to be processed in an installation implementing the process according to the invention.

Although not shown, an installation can continuously implement the process according to the invention, that is, at the start of the process from a feed hopper into which fresh algae is poured up to the tank/container for storing and collecting the lacto-fermented algae and fermented juice obtained at the end of the process. For example, one or more conveying devices, notably with belts, can be arranged in the installation between two stations for carrying out separate steps of the process.

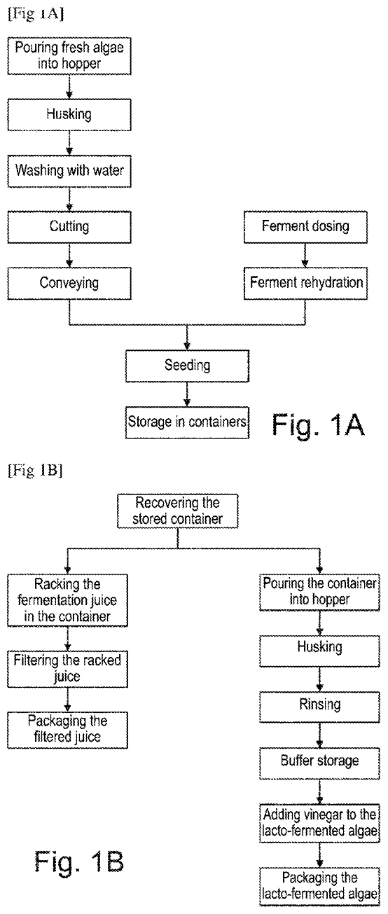

An example of the process according to the invention is now described in relation to FIGS. 1A and 1B.

Step i): Marine algae arrives fresh and unrinsed in the installation, directly from the place of harvesting thereof in the marine environment. One of the inherent advantages of the process is that pre-transport does not need to be carried out using a refrigerated vehicle. Of course, it can still be frozen at the start of the process, notably if it has been harvested for some time before the process according to the invention is implemented.

This fresh algae is poured into a feed hopper which can be standard.

During this step i), it is possible to perform a husking step i1) in a husker which may be standard. This husking makes it possible to regulate the feed rate of an algae cutting machine used afterwards and to proceed where applicable with a visual sorting of any undesirable elements, such as parasitic algae, shellfish optionally present in the algae such as periwinkles or others.

Step ii): The algae is then washed. This washing which consists of rinsing with fresh water removes any traces of sand from the algae. Preferably, this washing can be carried out within the husker itself.

Step iii): The algae is cut to a calibrated length.

Upstream of the cutting machine, a conveyor belt with a gentle incline can be used to pour the algae and feed it directly to the machine. This prevents the algae leaving the husker from agglomerating/clumping, that is, mixing together and becoming denser which can adversely affect the quality (homogeneity, precision) of the cut.

The cutting length performed is carefully selected based on the type of algae to be fermented.

Indeed, the cut performed increases the contact surface between the algae and the lactic ferments used while preserving the morphological properties of the algae that are important for its qualities as food.

For all marine algae known to the inventor, a cutting length of between 0.2 and 5 cm is currently envisaged.

Step iii1): Once the cut has been made, the cut algae is conveyed into at least one tank or container which serves as a storage, inoculation and collection area.

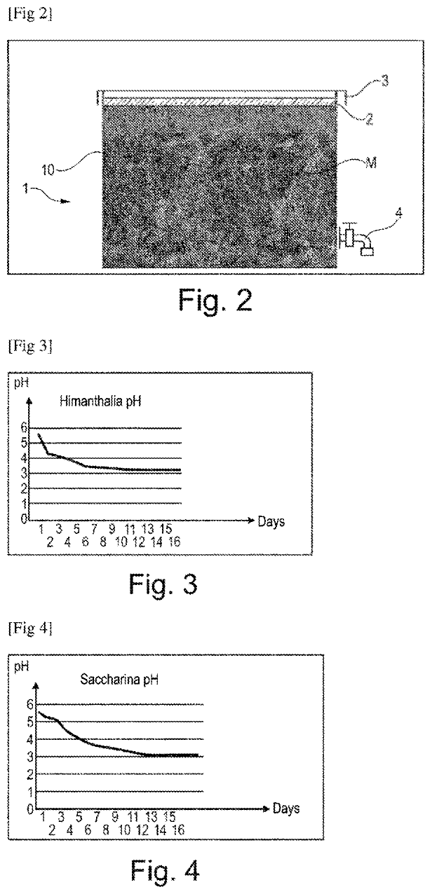

An example of a storage, inoculation and collection container is exemplified in FIG. 2.

This container 1 comprises a tank 10 internally delimiting the volume in which a mixture M of cut algae and water, then with ferments according to the subsequent lacto-fermentation step, is stored. The tank 10 can be a rigid plastic box with side walls and a watertight base, such as that marketed under the trade designation Geobox®.

The volume of mixture present in the tank 10 is topped by a plate 2 which forms a ballast on the free surface. This plate 2 is advantageously made of a non-oxidizing material. At the stage of this step iii), to create surface ballast, the tank 10 is preferably filled with water to around 20-40% of the weight of the stored algae. The ballast plate 2 further forms a sealing layer which prevents surface oxidation.

Additionally, the tank 10 is preferably sealed above the plate 2 by a cover 3. Advantageously, a watertight film can be applied to the cover 3 to limit gas exchange with the outside.

A tap 4 is arranged at the bottom of the tank 10 to draw off the lacto-fermented juice at the end of the process by gravity or by pumping.

Step iv): In parallel with the cutting process, an inoculum is prepared comprising predominantly lactic acid-producing bacteria.

To do this, ferments are dosed and rehydrated.

For example, the ferments are diluted in physiological water for 30 min at 20/25° C. to rehydrate them and to obtain adequate distribution.

When ferments are provided completely dehydrated initially, they are rehydrated for 1 hour in fresh water with 0.9% salt to avoid osmotic shock.

Several ferments, including Lactobacillus plantarum, Leuconostoc mesenteroides, Lactobacillus lactis, Lactobacillus zeae, Lactobacillus casei or paracasei, Lactobacillus harbinensis, Leuconostoc kimchii, Lactobacillus delbrueckii, Lactobacillus rhamnosus, Lactobacillus harbinensis and Streptococcus thermophilus, can be used and mixed together beforehand.

Step v): The algae can then be inoculated in a container such as the one detailed with reference to FIG. 2.

To do this, the inoculum provided in step iv) is mixed with the algae stored in the container and water to obtain the mixture M to be lacto-fermented.

The inoculum is dosed to obtain a concentration of lactic acid bacteria sufficient for the growth of the bacteria present in the algae so as to achieve lacto-fermentation thereof. The inoculation rate is between 10⁵ and 10⁷ CFU/g. For Himanthalia elongata, a particularly suitable strain is Lactobacillus plantarum at an inoculation rate of 10⁶ CFU/g. Typically, the mixture M in a given container contains cut algae, ferments rehydrated according to step iv) and salt, preferably in a proportion of 1 to 3% by weight of the algae and non-chlorinated fresh water, preferably in a proportion of 10 to 100% by weight of the algae.

This inoculation step can take place very quickly after harvesting fresh algae, typically between 24 and 48 hours after harvesting.

According to the invention, lacto-fermentation is regularly monitored and it is continued until the measured pH of the lacto-fermented composition has fallen below a predetermined final threshold. Preferably, this pH should be less than 4.5, preferably less than 4.2. Above a pH of around 4.2, 4.3, there may be a risk of pathogenic bacteria and where applicable, unrevealed palatability problems.

FIG. 3 shows the pH evolution of a mixture with Himanthalia elongata algae, cut to a length of between 0.5 and 1.3 cm.

This curve shows that the pH is induced by lacto-fermentation and it stabilizes over time.

Thus, the lacto-fermentation time according to the invention is considered complete when the pH is stabilized below a threshold, in order to be able to offer the algae as a food product.

The process can be optimized to lower and stabilize the pH more quickly for the same species of algae.

Typically, the choice of bacterial strains determines the pH level. The level of anaerobicity also has an influence, leading to the choice of a suitable storage container.

By way of example, as shown in FIG. 3, for Himanthalia elongata algae, when the pH is stabilized between 3.8 and 4.2, then the algae can be collected by separating them from the lacto-fermented juice as described below.

According to the process of the invention, the lacto-fermentation time which is necessary also depends on the texture of the algae required for subsequent consumption thereof.

Table 1 below summarizes the inventor's feedback on the texture for two algae species over time.

TABLE 1

| | Length of lacto- | |
|---|---|---|
| Algae species | fermentation step v) | Texture |
| Himanthalia elongata | 3 to 5 weeks | firm |
| (sea beans) | 5 to 8 weeks | tender |
| | 8 to 20 weeks | soft |
| Saccharina lactissima | 1 to 3 weeks | firm |
| (Royal Kombu) | 3 to 5 weeks | tender |
| | More than 5 weeks | soft |

This lacto-fermentation step v) is stopped when the algae are to be collected for packaging.

Step vi): prior to collecting the algae, at least some of the lacto-fermented juice obtained is collected.

The juice is separated from the algae after the pH of the juice has been checked again. Color, odor and viscosity are also indicators of the fermentation performed.

Preferably, the juice is collected by racking by gravity or pumping notably from the bottom of the tank 10 via the tap 3.

Typically, between 5% and 40% of the total volume of the mixture M is racked in the form of fermentation juice.

Preferably, this withdrawn volume is replaced by a substantially identical volume of water whose pH remains at the predetermined threshold.

Step vi1): Before storage thereof, the racked lacto-fermented juice is filtered to retain suspended organic elements. For example, it is possible to use a standard stainless steel filter with a very fine mesh, typically between 0.5 and 0.8 mm. A suitable filtration system can also be envisaged to recover the specific active molecules in the juice.

Step vi2): The filtered juice is then packaged. This can be done in small cans or buckets, typically 20 L or in larger drums, typically 220 L. It is also possible to package the juice in IBCs (Intermediate Bulk Containers) which are tank containers with a large unit volume, typically 1000 L, and which are usually used for storing and transporting liquids.

The storage time can be up to an expiry date of 6 months at room temperature, for the tests carried out on the algae species tested. It is even possible to envisage a storage time up to 12 months in suitable barrels.

The juice collected and stored can be used directly or indirectly for animal, plant or human consumption.

The inventor has carried out tests on the juice obtained for different species of algae.

The juice obtained from Laminaria digitata is highly foaming with probable applications in cosmetics and in human nutrition.

The juice obtained from Ulva lactuca (sea lettuce) is foaming with probable applications as a palatability enhancer and food supplement in animal nutrition.

The juice obtained from Palmaria palmata has a very intense violet color with probable applications as a natural colorant.

Lacto-fermented algae and the juice obtained from Himanthalia elongata (sea beans), Saccharina lactissima (Royal Kombu) and Laminaria digitata have antioxidant, emulsifying and aromatic properties. For the antioxidant properties to be brought out, the inventor believes that the juice should be concentrated.

Step vii): once the lacto-fermented juice has been racked, the lacto-fermented algae is washed, stored in a buffer tank and collected.

More precisely, the lacto-fermented algae is poured into a hopper for light rinsing with fresh water, preceded, if necessary, by husking. It is then drained into a container. This can be a GEABOX® type container with perforated walls. This draining process removes as much lactic acid as possible from the algae to interrupt the fermentation process as far as possible. Preferably, during fermentation, algae can be mixed into the juice for anaerobic homogenization, followed by transfer to a container acting as buffer storage.

To stabilize the algae and therefore prevent the growth of harmful bacteria, 5% organic white vinegar in fresh water or 0.8% citric acid can be added to the packaging volume to achieve a pH of 3 and to keep the packaged algae intact for a minimum period of 6 months at room temperature, which defines its expiry date. Instead of water, a liquid with a pH between 2 and 3 can be used to obtain the stability sought for packaging.

Algae can be packaged in small cans or buckets, typically 10 L or in larger drums, typically 220 L.

As an example of the process described above, Himanthalia elongata algae was cut to a length of between 0.5 and 1.3 cm.

This was followed by salting in a quantity of 1 to 10% with respect to the weight of the cut algae.

The cut and salted algae was stored at a temperature of between 1° and 30° C. in closed tanks and individually weighted by a plate weighing around 10% of the weight of the algae contained in a tank.

Inoculation was carried out at this room temperature sequentially as follows:
inoculating for primary heterolactic fermentation with a mixture of 5×10⁵ Leuconostoc mesenteroides per gram of algae and 5×10⁵ Lactobacillus brevis per gram of algae;
measuring the acidity of the mixture down to a pH of around 4.5;
inoculating for secondary homolactic fermentation using Lactobacillus plantarum in a quantity of 10⁶ germs per gram of algae until pH 4 is reached, which corresponds to a duration of approximately 6 days.

The inventor carried out further tests.

At the end of the juice racking step, the lacto-fermented algae collected and therefore separated from the juice was pressed and dried in a dryer.

This drying can be carried out at a temperature range of 40° C. to 100° C.

The dried algae was then ground/micronized to obtain lacto-fermented algae powders with effective functional and nutritional properties.

Tests with lacto-fermented sea beans dried to 88% dry matter and micronized to a size of between 0.3 and 0.8 mm show a water absorption capacity of a factor of 15.

This natural biodegradable absorbent consisting of dried and micronized algae may represent an interesting plant-based alternative to food additives (Carboxymethylcellulose (E466), Sodium alginate (E401), Pectin (E440), Gum arabic (E414), Guar gum (E412), Modified starch (E1404, E1412, E1414), Carrageenan (E407), Alginic acid (E400)). These additives are also used in various industries (cosmetics, hygiene products, etc.)

Other alternatives and improvements can be envisaged without going beyond the scope of the invention.

If, in the example exemplified, the container is used simultaneously for the steps of storing the algae prior to inoculation, for lacto-fermentation and for collection, it may be envisaged to have one or more tanks for storing algae prior to the inoculation thereof, one or more other tanks for lacto-fermentation and for collecting the lacto-fermented algae and juice.

As lacto-fermentation containers, IBC-type tanks can be used, in a version equipped with a valve or tap for racking the lacto-fermented juice by gravity or pumping.

The example shown hereinbefore corresponds to Himanthalia elongata with inoculation.

For certain species of algae, it is possible to dispense with the inoculation step and therefore with the prior dosing then rehydration of ferments.

Indeed, natural lacto-fermentation may suffice between the cutting step and the container storage step as according to FIG. 1A.

This natural lacto-fermentation can relate for example to Royal Kombu for which fermentation takes place with endogenous bacterial flora.

FIG. 4 shows a curve showing the pH of the exclusively natural fermentation of Royal Kombu during a process just described. The juice is also separated from the algae after checking that the stabilized pH of the juice is less than 3.2. Color, odor and viscosity are also indicators of the maturity of this algae stored in fermentation.

For the lacto-fermentation of Ulva lactuca (sea lettuce), it is possible to use an open tank for oxygenation and if necessary stirring with a bubbler arranged within the fermentation mixture volume. Seawater or 3.5% salted fresh water can be used for preservation. To obtain a lacto-fermented juice with suitable properties, fresh water is preferred to obtain an osmotic shock and tenderize the sea lettuce to promote the action of lactic acid bacteria and thus dissolve the algae with little or no production of H₂S gas. For this lacto-fermentation of Ulva lactuca, the inventor has tested Lactobacillus harbinensis and Leuconostoc kimchii as promising strains. The juice is separated from the algae after checking that the stabilized pH of the juice is less than 4.2. Color, odor and viscosity are also indicators of the maturity of this algae stored in fermentation. For Ulva lactuca, the inventor plans to mix the algae and the juice obtained to form a nutritional purée.

In addition to or instead of a sealing plate above the mixture undergoing lacto-fermentation, a gas such as CO₂ or nitrogen or other can be circulated over the free surface of the mixture.

Within the scope of the invention, various types of equipment can be tested and implemented to optimize the extraction, separation and concentration of the co-products obtained (lacto-fermented juices and algae). For example, the juices can be concentrated and/or filtered by various mechanical systems (atomization, ultrafiltration, centrifugal, tangential etc.) The choice of system preferably depends on the degree of transformation of the active ingredients desired to be obtained. The active ingredients present in lacto-fermented juices vary based on the varieties and strains of ferments used.

Furthermore, enzymes can also be added, notably when the variety of algae makes the action of inoculating bacteria or fermentation strains difficult. For example, endoglucanases and pectinases can be added to transform green algae: due to their rapid action, these enzymes can release the elements needed by lactic acid bacteria and initiate fermentation without producing hydrogen sulfide (H₂S). Enzymes with specific actions can also be considered to improve the quality of lacto-fermented juices. For example, these specific enzymes can lower the heavy metal content of the juices.

To lower the heavy metal content of the juices, the use of microalgae in a photoreactor can also be envisaged.

In addition to the applications already recited for the algae and lacto-fermented juice collected at the end of the process, a wide range of uses can be envisaged, as follows.

### Lacto-Fermented Algae:

A/Human food:

Manufacturers are increasingly seeking plant-based alternatives to animal proteins and are reducing or even replacing chemical additives with natural extracts.

The inventor carried out tests by mixing 30% lacto-fermented sea beans obtained according to the process of the invention, with ground beef in order to obtain a steak.

These tests showed that the nutritional value of the steak, characterized in France by the Nutri-Score, was improved, without altering the taste and moreover reducing production costs.

The inventor also carried out tests by mixing lacto-fermented sea beans obtained according to the process of the invention, with sausage meat.

These tests showed that the mixtures produced had preservative properties that make them credible alternatives to the nitrite salts currently used.

### B/Animal Feed, Notably for Sheep, Cattle, Pigs or Poultry:

Lacto-fermented algae obtained according to the process of the invention can be used as a nutritional raw material, flavoring factor, texturizer or absorbent. For example, lacto-fermented algae can be mixed with cattle feed to improve the balance of their microbiota and the performance of their digestive metabolism and therefore to improve the quantity and quality of the milk they produce.

Also, it is possible to envisage adding lacto-fermented algae obtained according to the process of the invention in doses of medication, notably in boluses: indeed, the texturizing and nutritional qualities of algae can improve the effects of the medication on the animal.

It is also possible to envisage the use of lacto-fermented algae, notably in a moist state, as part of an animal feed ration, to improve the quality of the meat (omega-3 fatty acids).

Moreover, the inventor has carried out encouraging tests, by mixing lacto-fermented algae collected according to the process of the invention to obtain an optimum density of substrates for insect larvae.

Further tests are currently being carried out with lacto-fermented algae, dried and micronized according to the process of the invention which have been mixed with aquaculture feed (fish meal) to offer a bioavailable plant alternative and improve fish flesh quality, farm water quality and reduce mortality.

Finally, for plant biostimulation, the inventor believes that different varieties of lacto-fermented algae obtained according to the process of the invention should be tested by mixing them with biochar, with the aim of improving growth through water regulation and the availability of nutrients required by plants and soil organisms.

### Lacto-Fermented Juices:

### A/Human Food:

The inventor believes that mixing a lacto-fermented juice obtained according to the invention into a sports drink can increase metabolite intake.

Tests are forthcoming to use the juice obtained according to the invention in hams to reduce or even replace nitrite salts.

Additionally, depending on the concentration of emulsifying active ingredients, the juices obtained according to the invention can be alternatives to egg whites such as aquafaba (chickpea juice) which is often used in cooking and in baking, particularly in vegan and vegetarian diets as a substitute by virtue of its ability to foam and to act as a binder. The inventor also believes that topping with a juice obtained according to the invention to achieve a vegan decoration on buns instead of the usual egg white, should be envisaged. The inventor also plans to test the juices obtained according to the invention in breads and cookies to replace additives and improve texture and/or crispness and avoid breakage in cookie making.

### B/Animal Feed, Notably for Sheep, Cattle, Pigs or Poultry:

The inventor believes that a lacto-fermented juice obtained according to the invention, as a beverage, should make it possible to reduce the thermal stress of animals when temperatures are high on livestock farms. Animals can reduce their feed consumption and this negatively affects growth, milk production, meat quality, reproduction and general health. A lacto-fermented juice obtained according to the invention, with its potential for hydration and palatability is a solution whose efficacy should be tested directly on the farm.

Another envisaged use for a lacto-fermented juice obtained according to the invention is to hydrate poultry while avoiding biofilm formation in liquid feed systems, due to the antibacterial properties of the juice.

Finally, the inventor believes that other advantages could also be provided by lacto-fermented algae juices obtained according to the invention, including:
improved animal growth and performance, through the supply of essential nutrients such as amino acids, vitamins and minerals,
a boost to the immune system, through the supply of certain algae-specific polysaccharides which have immunostimulant properties,
reduced oxidative stress,
improved digestive health especially with probiotics and postbiotics derived from lacto-fermentation which act favorably on intestinal flora.

1. A process for transforming algae comprising the following steps:
i) providing algae harvested from aquatic environments, frozen if necessary;
ii) washing the algae provided according to step i) with water;
iii) optionally, cutting or grinding the algae washed according to step ii);
iv) optionally, providing an inoculum comprising predominantly lactic acid-producing bacteria;
v) either inoculating the algae optionally cut or ground according to step iii) with the inoculum provided according to step iv) which comprises a concentration of lactic acid bacteria sufficient for the growth of the bacteria present in the algae so as to perform the lacto-fermentation thereof, or natural lacto-fermentation of the algae until the measured pH of the lacto-fermented composition has fallen below a predetermined final threshold;
vi) collecting at least part of the lacto-fermented juice obtained;
vii) washing the lacto-fermented algae, followed by storage and collection thereof.

2. The process according to claim 1, the algae provided in step i) being marine algae selected from one of the following species: Gracilaria verrucosa, Chondrus crispus, Himanthalia elongata, commonly known as sea beans, Laminaria digitata, commonly known as Breton kombu seaweed, Undaria pinnatifida, Palmaria palmata, Ulva, Solieria chordalis, Porphyra umbilicalis, Fucus visiculosus, Ascophyllum nodosum, Saccharina japonica, commonly known as kombu seaweed, Saccharina latissima, commonly known as royal kombu seaweed, Ulva lactuca commonly known as sea lettuce or a combination thereof.

3. The process according to claim 1, step iii) being carried out so that the length of the cut algae is between 0.2 and 5 cm.

4. The process according to claim 1, the lactic acid bacteria being selected from one of the following species: Lactobacillus plantarum, Leuconostoc mesenteroides, Lactobacillus lactis, Lactobacillus zeae, Lactobacillus casei or paracasei, Lactobacillus harbinensis, Leuconostoc kimchii, Lactobacillus delbrueckii, Lactobacillus rhamnosus, Lactobacillus harbinensis, Streptococcus thermophilus or a combination thereof.

5. The process according to claim 1, with inoculation step v) performed by a combination of Leuconostoc mesenteroides and Lactobacillus lactis or zeae.

6. The process according to claim 5, the inoculation rate being between 10⁵ and 10⁷ CFU/g.

7. The process according to claim 1, comprising prior to the inoculation step v), packaging the cut or ground algae in a storage tank followed by anaerobic digestion thereof.

8. The process according to claim 1, collection step vi) being carried out by racking the lacto-fermented juice.

9. The process according to claim 8, comprising prior to step vii), a step of adding a liquid selected from water, water and acid or a combination thereof, the volume of liquid added preferably being substantially equal to that of the racked lacto-fermented juice.

10. The process according to claim 9, comprising, once the liquid has been added and before racking, a maceration step lasting between 1 and 20 days, preferably between 1 and 15 days.

11. The process according to claim 1, the predetermined final pH threshold is less than or equal to 4.5.

12. The use of lacto-fermented algae collected in step vii) of the process according to claim 1, for human consumption.

13. The use of the lacto-fermented juice collected in step vi) of the process according to one of claim 1, as a palatability enhancer for animal feed, notably for sheep, cattle or pigs or poultry, or as a colorant, flavoring and/or foaming agent, or emulsifier in human food or animal feed and in cosmetics.

14. The use of the lacto-fermented juice collected in step vi) of the process according to claim 1, where applicable mixed with lacto-fermented algae collected in step vii) of the process according to claim 1 as a phyto-stimulant for plant production and/or for improving soil quality.

15. The lacto-fermented juice collected in step vi) of the process according to claim 1, for use thereof in pharmacology.
